(19) United States
(12) Patent Application Publication
(10) Pub. No.: US 2024/0363196 A1
(43) Pub. Date: Oct. 31, 2024

(54) SYSTEM AND METHOD FOR IDENTIFYING MOLECULAR PATHWAYS PERTURBED UNDER INFLUENCE OF DRUG OR DISEASE

(71) Applicant: Innoplexus AG, Eschborn (DE)

(72) Inventor: Om Sharma, Pimpri-Chinchwad (IN)

(73) Assignee: Innoplexus AG, Eschborn (DE)

(21) Appl. No.: 18/308,191

(22) Filed: Apr. 27, 2023

(51) Int. Cl. G16B 25/10; G16B 40/20

(52) U.S. Cl. CPC G16B 25/10; G16B 40/20

(57) ABSTRACT

A method for identifying molecular pathways perturbed under influence of a drug or a disease includes extracting a relationship dataset related to genes and molecular pathways associated with the genes, from a pre-curated database. The method further includes mapping the relationship dataset onto a research graph. The method further includes identifying, sub-networks within the research graph, and assigning a gene score to each gene in the identified sub-networks, based on whether a gene is neutral, dysregulated, or associated with a disease-specific organ. The method further includes determining a perturbed molecular pathway for genes within the identified sub-networks based on the gene score and a molecular pathway interconnectivity within the research graph. The perturbed molecular pathway for genes has a highest association with a pathophysiology of the disease or the drug response as compared to other molecular pathways associated with the genes in the research graph.

### FIELD OF TECHNOLOGY

The present disclosure relates generally to a field of computational biology and bioinformatics, and more specifically, to a system and a method for identifying molecular pathways perturbed under influence of drug or disease.

### BACKGROUND

In order to understand disease pathophysiology and develop effective therapeutic strategies, it is important to identify perturbed pathways in a given disease. However, the identification of such pathways is a complex and challenging task due to the involvement of various genes and molecular pathways in a disease.

Conventionally, there are several types of approaches for pathway prioritization, including Bayesian, Signaling pathway impact analysis, Gene Graph Enrichment Analysis, Topology-based pathway analysis, and Over-representation Analysis. However, each of such approaches may have demerits such as limited accuracy, complexity, and high computational requirements. The existing approaches for pathway prioritization have several limitations and may not provide a complete understanding of disease pathophysiology. Therefore, there is a need for a new and improved method for identifying the most crucial pathways in a given disease.

Further limitations and disadvantages of conventional and traditional approaches will become apparent to one of skill in the art through comparison of such systems with some aspects of the present disclosure, as set forth in the remainder of the present application with reference to the drawings.

### BRIEF SUMMARY OF THE DISCLOSURE

The present disclosure provides a system and a method for identifying molecular pathways perturbed under influence of a given drug or a disease of interest. The present disclosure seeks to provide a solution to the existing problem of identifying the most crucial molecular pathways for a given drug or target or disease of interest. According to conventional understanding, various genes may participate in disease pathophysiology through molecular pathways, but determining the magnitude of pathway influence or change may be challenging. An aim of the present disclosure is to provide a solution that overcomes at least partially the problems encountered in prior art and provide an improved method and system for identifying the molecular pathways perturbed under influence of a given drug or disease by using a research graph.

In one aspect, the present disclosure provides a method for identifying molecular pathways perturbed under influence of a given drug or a disease of interest, the method comprising:
extracting, by a processor, a relationship dataset related to a plurality of genes and one or more molecular pathways associated with the plurality of genes, from a pre-curated database;
mapping, by the processor, the relationship dataset onto a research graph, wherein the research graph is indicative of a molecular pathway connectivity of the plurality of genes in a pathophysiological condition;
identifying, by the processor, one or more sub-networks within the research graph, wherein each sub-network comprises a start gene, one or more end genes, and one or more molecular pathways associated with the start gene and the one or more end genes within the research graph;
assigning, by the processor, a gene score to each gene in the one or more identified sub-networks, based on whether a gene is neutral, dysregulated, or associated with a disease-specific organ; and
determining, by the processor, a perturbed molecular pathway for genes within the one or more identified sub-networks based on the gene score and a molecular pathway interconnectivity within the research graph,
wherein the perturbed molecular pathway for genes is a molecular pathway which has a highest association with a pathophysiology of the disease of interest or the drug response of the given drug as compared to other molecular pathways associated with the plurality of genes in the research graph, and
wherein the determined perturbed molecular pathway is utilized to remediate one or more of: the disease of interest and the drug response of the given drug.

The method of the present disclosure utilizes the pre-curated database and the research graph to efficiently identify the molecular pathways perturbed by the given drug or disease. This reduces the time and resources required for identifying perturbed molecular pathways compared to traditional experimental approaches. By assigning gene scores and considering molecular pathway interconnectivity within the research graph, the method accurately determines the perturbed molecular pathway. This ensures that the remediation strategies are focused on the most relevant molecular pathways, increasing the likelihood of success. Further, the method identifies the molecular pathways perturbed under the influence of a specific disease or drug, allowing for personalized treatment options. This can lead to improved patient outcomes and reduced healthcare costs. The method may also be applied to a wide range of diseases and drugs, making it a versatile tool for drug discovery and personalized medicine.

In another aspect, the present disclosure provides a system for identifying molecular pathways perturbed under influence of a given drug or a disease of interest, the system comprising:
a processor configured to:
   extract a relationship dataset related to a plurality of genes and one or more molecular pathways associated with the plurality of genes, from a pre-curated database;
   map the relationship dataset onto a research graph, wherein the research graph is indicative of a molecular pathway connectivity of the plurality of genes in a pathophysiological condition;
   identify one or more sub-networks within the research graph, wherein each sub-network comprises a start gene, one or more end genes, and one or more molecular pathways associated with the start gene and the one or more end genes within the research graph;
   assign a gene score to each gene in the one or more identified sub-networks, based on whether a gene is neutral, dysregulated, or associated with a disease-specific organ; and
   determine a perturbed molecular pathway for genes within the one or more identified sub-networks based on the gene score and a molecular pathway interconnectivity within the research graph,
   wherein the perturbed molecular pathway for genes is a molecular pathway which has a highest association with a pathophysiology of the disease of interest or the drug response of the given drug as compared to other molecular pathways associated with the plurality of genes in the research graph, and
   wherein the determined perturbed molecular pathway is utilized to remediate one or more of: the disease of interest and the drug response of the given drug.

The system achieves all the advantages and technical effects of the method of the present disclosure.

It has to be noted that all devices, elements, circuitry, units and means described in the present application could be implemented in the software or hardware elements or any kind of combination thereof. All steps which are performed by the various entities described in the present application as well as the functionalities described to be performed by the various entities are intended to mean that the respective entity is adapted to or configured to perform the respective steps and functionalities. Even if, in the following description of specific embodiments, a specific functionality or step to be performed by external entities is not reflected in the description of a specific detailed element of that entity which performs that specific step or functionality, it should be clear for a skilled person that these methods and functionalities can be implemented in respective software or hardware elements, or any kind of combination thereof. It will be appreciated that features of the present disclosure are susceptible to being combined in various combinations without departing from the scope of the present disclosure as defined by the appended claims.

Additional aspects, advantages, features, and objects of the present disclosure would be made apparent from the drawings and the detailed description of the illustrative implementations construed in conjunction with the appended claims that follow.

### BRIEF DESCRIPTION OF THE DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those in the art will understand that the drawings are not to scale. Wherever possible, like elements have been indicated by identical numbers.

Embodiments of the present disclosure will now be described, by way of example only, with reference to the following diagrams wherein:

FIG. 1 is a block diagram of a system for identifying molecular pathways perturbed under influence of a given drug or a disease of interest, in accordance with an embodiment of the present disclosure;

FIG. 2 is a flowchart to rank the molecular pathways based on perturbation score, in accordance with an embodiment of the present disclosure;

FIG. 3 is an exemplary research graph for a given set of genes, in accordance with an embodiment of the present disclosure; and

FIG. 4 is a flowchart of a method for identifying molecular pathways perturbed under influence of the given drug or the disease of interest, in accordance with an embodiment of the present disclosure.

In the accompanying drawings, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the non-underlined number is used to identify a general item at which the arrow is pointing.

### DETAILED DESCRIPTION OF THE DISCLOSURE

The following detailed description illustrates embodiments of the present disclosure and ways in which they can be implemented. Although some modes of carrying out the present disclosure have been disclosed, those skilled in the art would recognize that other embodiments for carrying out or practicing the present disclosure are also possible.

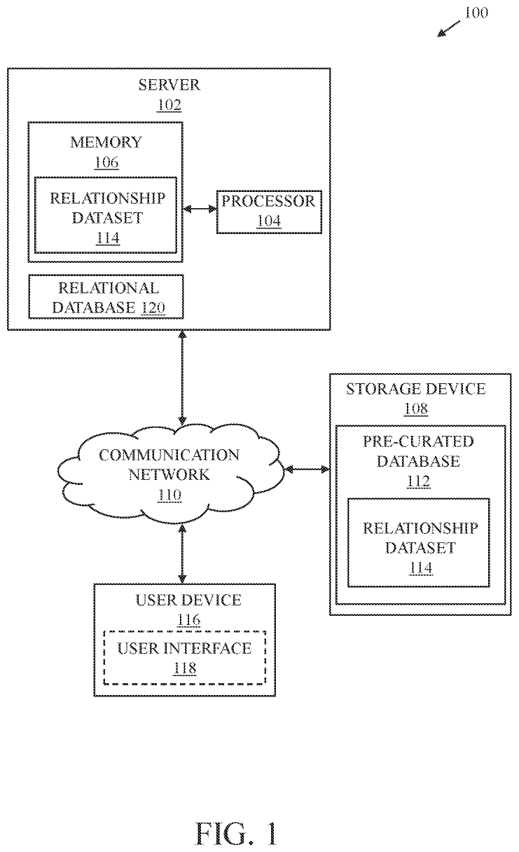

FIG. 1 is a block diagram of a system for identifying molecular pathways perturbed under influence of a given drug or a disease of interest, in accordance with an embodiment of the present disclosure. With reference to FIG. 1, there is shown a block diagram of a system 100. The system 100 includes a server 102, a processor 104, and a memory 106. The processor 104 is communicatively coupled with the memory 106. The system 100 may be used to identify molecular pathways that are perturbed under influence of a given drug or a disease of interest.

In an implementation, the processor 104 and the memory 106 may be implemented on a same server, such as the server 102. In some implementations, the system 100 further includes a storage device 108 communicatively coupled to the server 102 via a communication network 110. The storage device 108 includes a pre-curated database 112. In some implementations, the pre-curated database 112 includes a relationship dataset 114 that may be retrieved from the storage device 110 by the memory 106, as per requirement. The relationship dataset 114 includes a plurality of genes and one or more molecular pathways associated with the plurality of genes. In some implementations, the pre-curated database 112 may be stored in the same server, such as the server 102. In some other implementations, the pre-curated database 112 may be stored outside the server 102, as shown in FIG. 1. The server 102 may be communicatively coupled to a plurality of user devices, such as a user device 116, via the communication network 112. The user device 116 includes a user interface 118.

The present disclosure provides the system 100 that identifying molecular pathways perturbed under influence of the given drug or the disease of interest, where the system 100 determines a perturbed molecular pathway for genes using a research graph. The perturbed molecular pathway refers to a molecular pathway that is significantly altered or disrupted in response to a specific stimulus, such as the drug treatment or a disease condition. In some implementations, the perturbed molecular pathway determined by the system 100 refers to a molecular pathway which has a highest association with a pathophysiology of the disease of interest or the drug response of the given drug as compared to other molecular pathways associated with the plurality of genes in the research graph. In other words, the perturbed molecular pathway determined by the system 100 refers to a molecular pathway that is most significantly altered or disrupted in response to the specific stimulus, such as the drug treatment or a disease condition, as compared to the other molecular pathways associated with the plurality of genes in the research graph. The research graph refers to a graph that represents the molecular pathway connectivity of a set of genes in a pathophysiological condition. The nodes of the graph represent genes or proteins, and the edges represent the molecular interactions or relationships between them.

The server 102 includes suitable logic, circuitry, interfaces, and code that may be configured to communicate with the user device 116 via the communication network 110. In an implementation, the server 102 may be a master server or a master machine that is a part of a data center that controls an array of other cloud servers communicatively coupled to it for load balancing, running customized applications, and efficient data management. Examples of the server 102 may include, but are not limited to a cloud server, an application server, a data server, or an electronic data processing device.

The processor 104 refers to a computational element that is operable to respond to and processes instructions that drive the system 100. The processor 104 may refer to one or more individual processors, processing devices, and various elements associated with a processing device that may be shared by other processing devices. Additionally, the one or more individual processors, processing devices, and elements are arranged in various architectures for responding to and processing the instructions that drive the system 100. In some implementations, the processor 104 may be an independent unit and may be located outside the server 102 of the system 100. Examples of the processor 104 may include but are not limited to, a hardware processor, a digital signal processor (DSP), a microprocessor, a microcontroller, a complex instruction set computing (CISC) processor, an application-specific integrated circuit (ASIC) processor, a reduced instruction set (RISC) processor, a very long instruction word (VLIW) processor, a state machine, a data processing unit, a graphics processing unit (GPU), and other processors or control circuitry.

The memory 106 refers to a volatile or persistent medium, such as an electrical circuit, magnetic disk, virtual memory, or optical disk, in which a computer can store data or software for any duration. Optionally, the memory 106 is a non-volatile mass storage, such as a physical storage media. The memory 106 is configured to store the relationship dataset 114. Furthermore, a single memory may encompass and, in a scenario, and the system 100 is distributed, the processor 104, the memory 106 and/or storage capability may be distributed as well. Examples of implementation of the memory 106 may include, but are not limited to, an Electrically Erasable Programmable Read-Only Memory (EEPROM), Dynamic Random-Access Memory (DRAM), Random Access Memory (RAM), Read-Only Memory (ROM), Hard Disk Drive (HDD), Flash memory, a Secure Digital (SD) card, Solid-State Drive (SSD), and/or CPU cache memory.

The storage device 108 may be any storage device that stores data and applications without any limitation thereto. In an implementation, the storage device 108 may be a cloud storage, or an array of storage devices.

The communication network 110 includes a medium (e.g., a communication channel) through which the user device 116 communicates with the server 102. The communication network 110 may be a wired or wireless communication network. Examples of the communication network 110 may include, but are not limited to, Internet, a Local Area Network (LAN), a wireless personal area network (WPAN), a Wireless Local Area Network (WLAN), a wireless wide area network (WWAN), a cloud network, a Long-Term Evolution (LTE) network, a plain old telephone service (POTS), a Metropolitan Area Network (MAN), and/or the Internet.

The pre-curated database 112 includes various types of biological data, such as gene expression data, protein-protein interaction data, signaling pathways, genetic variants, and disease information. The pre-curated database 112 also includes information about the quality and reliability of the data, as well as annotations and metadata that help to identify and interpret the data. Data in the pre-curated database 112 may come from various sources, such as public repositories, scientific literature, and experimental data generated by researchers.

The relationship dataset 114 includes information about the relationships or interactions between different entities, such as genes, proteins, pathways, or diseases. The relationship dataset 114 further includes information such as protein-protein interactions, gene regulatory networks, metabolic pathways, disease-gene associations, and drug-target interactions, among others. The relationship dataset 114 may further include various types of annotations and metadata to provide additional context and information about the relationships.

The user device 116 refers to an electronic computing device operated by a user. In an implementation, the user device 116 may be configured to obtain a user input of a given set of input genes in a search portal or a search engine rendered over the user interface 118 and communicate the user input to the server 102. The server 102 may then be configured to retrieve the perturbed molecular pathways of the given set of input genes. Examples of the user device 116 may include but not limited to a mobile device, a smartphone, a desktop computer, a laptop computer, a Chromebook, a tablet computer, a robotic device, or other user devices.

In operation, the processor 104 is configured to extract the relationship dataset 114 related to the plurality of genes and the one or more molecular pathways associated with the plurality of genes, from the pre-curated database 112. For example, a relationship dataset is extracted that is related to genes that are involved in various molecular pathways associated with different types of cancer such as EGFR signaling pathway, which is associated with several types of cancer, including lung cancer, breast cancer, and colon cancer, from a pre-curated database of gene-molecular pathway relationships related to cancer. To extract the relationship dataset related to the EGFR signaling pathway, the processor 104 may search the pre-curated database and extract information on each gene from the plurality of genes that are involved in the EGFR signaling pathway, along with their interactions and associations with other genes and molecules in the pathway. The relationship dataset may include information on the biological functions of the genes, their roles in the pathway, and the molecular mechanisms underlying their interactions.

The processor 104 is further configured to map the relationship dataset 114 onto the research graph. The research graph is indicative of a molecular pathway connectivity of the plurality of genes in a pathophysiological condition. In some examples, the pathophysiological condition refers a state of abnormal function or structure of an organ, system, or the body as a whole due to a disease, disorder, or injury. The pathophysiological condition may be a disease, disorder, or any other condition related to the molecular pathways being studied. For example, if the research is focused on cancer, then the pathophysiological condition may be a specific type of cancer, such as breast cancer or lung cancer. In some implementations, in order to map the relationship dataset 114 onto the research graph, the processor 104 is further configured to construct the research graph by representing each gene and the one or more associated molecular pathways as nodes and representing the relationships between the plurality of genes and the one or more associated molecular pathways as edges in the research graph.

The processor 104 is further configured to identify one or more sub-networks within the research graph. Each sub-network includes a start gene, one or more end genes, and one or more molecular pathways associated with the start gene and the one or more end genes within the research graph. In some implementations, the one or more identified sub-networks includes a homogeneous network, a heterogeneous network, a heterogeneous multi-layered network, or a combination thereof. In some other implementations, in order to identify the one or more sub-networks within the research graph, the processor 104 is further configured to identify one or more start genes within the research graph based on topological information and curated pathway data obtained from the curated database. The topological information may include network properties such as degree centrality, betweenness centrality, and closeness centrality, which provide information on the importance of a node within the network. The curated pathway data may include specific genes, proteins, or molecules involved in a pathway, their interactions, and their functions. After identifying the one or more start genes within the research graph, the processor 104 is further configured to identify the one or more end genes associated with each start gene by following the molecular connectivity in the research graph until a pathway endpoint is reached. After identifying the start and end genes, the processor 104 is further configured to group each start gene and the one or more associated end genes into the one or more sub-networks based on the molecular connectivity.

It should be noted that each sub-networks of the one or more sub-networks may include one or more consequent genes between the start and end genes. However, in some examples, some of the one or more sub-networks may include only start gene and the one or more associated end genes.

The processor 104 is further configured to assign a gene score to each gene in the one or more identified sub-networks, based on whether a gene is neutral, dysregulated, or associated with a disease-specific organ. In other words, the processor 104 is further configured to assign the gene score to each gene in the one or more identified sub-networks, based on gene expression status. The gene expression status is identified by gene expression data that is obtained from a patient sample.

In some implementations, in order to assign the gene score to each gene in the one or more identified sub-networks, the processor 104 is further configured to obtain the gene expression data from the patient sample, identify the gene expression status based on the gene expression data obtained from the patient sample, and assign the gene score based on the gene expression status. The gene expression data refers to the measurement of the amount or activity of a particular gene in a given cell or tissue sample of a patient. The gene expression data may provide insight into how genes are regulated and may be used to identify genes that are differentially expressed between different conditions or disease states. The gene expression status refers to the level of expression or activity of a particular gene in a cell or tissue sample. It indicates whether a gene is turned on or off, and to what extent. The gene expression status may be influenced by various factors such as developmental stage, environmental cues, and disease state. The measurement of the gene expression status may provide insight into the biological processes and pathways involved in normal and abnormal cellular functions. For example, if the gene is neither overexpressed nor under expressed in a specific condition, then the gene score assigned to the gene is zero (0). If the gene is either overexpressed or under expressed in a particular condition based on the gene expression data, then the gene score assigned to the gene is one (1). If the gene has a status where both the gene expression and protein expression are dysregulated in a specific condition, then the gene score assigned to the gene is two (2). If the gene has a status where an expression of the gene is specific to a particular organ affected by a disease, then the gene score assigned to the gene is three (3). In an example, when the gene score is three (3), in pancreatic cancer, the expression of certain genes is specific to the pancreas, while in colorectal cancer, the expression of certain genes is specific to the colon.

In accordance with an embodiment, the processor 104 is further configured to store the relationship dataset 114 and the gene score of each gene in a relational database 120 in a linear vector form for constructing the research graph.

In some implementations, the processor 104 is further configured to calculate a pathway perturbation score (P_{S}) for each molecular pathway within the one or more identified sub-networks based on the gene score and the molecular pathway interconnectivity within the one or more identified sub-networks. The pathway perturbation score (P_{S}) of the molecular pathways may be defined for the perturbed pathway for the disease or for the perturbed pathway for a given set of genes. The perturbed molecular pathway for the disease refers to the pathway that is most dysregulated or disrupted in the context of the disease. This can be determined by analyzing gene expression data or other types of molecular data from individuals with the disease compared to those without the disease. The pathway perturbation score (P_{S}) for each molecular pathway within the one or more identified sub-networks for the disease is defined by Equation 1 provided below.

$${P}_{s} = ( \frac{PC}{{G}^{Coexpression}} ) \{ ( {Ig}^{N} ) + ( {G}^{P} ) - ( {PC}^{N} ) \} \qquad Equation 1$$

where, Ig^{N} is a number of initial gene for the one or more molecular pathways, G^{P} is predecessors gene count of Ig^{N}, G^{Coexpression} is a number of dysregulated gene in a disease condition, PC^{N} is a number of the one or more identified sub-networks, and PC is a number of molecular pathway inter-connectivity of initial gene network.

On the other hand, the perturbed pathway for the given set of genes refers to the pathway that is most dysregulated or disrupted when considering only the genes in the set. This can also be determined by analyzing gene expression data or other molecular data specifically for the set of genes in question. The pathway perturbation score (P_{S}) for each molecular pathway within the one or more identified sub-networks for the given set of genes is defined by Equation 2 provided below.

$${P}_{s} = ( \frac{PC}{{G}^{OverlapGene}} ) \{ ( {G}^{S} ) + ( {G}^{P} ) - ( {PC}^{N} ) \} \qquad Equation 2$$

where, Ig^{N} is a number of initial gene for the one or more molecular pathways, G^{P} is predecessors gene count of Ig^{N}. G^{OverlapGene} is a number of overlapped gene in the research graph, PC^{N} is a number of the one or more identified sub-networks, PC is a number of molecular pathway inter-connectivity of initial gene network, and G^{S} is the gene score.

In some implementations, the processor 104 is further configured to rank the one or more associated molecular pathways within each identified sub-network based on the respective pathway perturbation score. It should be noted that a higher ranked pathway is more perturbed than a lower ranked pathway. The ranking is based on the level of dysregulation or perturbation observed in the genes or proteins involved in the molecular pathway. The higher ranked pathway refers to a pathway that is more significantly affected or perturbed in a particular condition or disease compared to other pathways.

The processor 104 is further configured to determine a perturbed molecular pathway for genes within the one or more identified sub-networks based on the gene score and a molecular pathway interconnectivity within the research graph. In some implementations, in order to determine the perturbed molecular pathway for genes within the one or more identified sub-networks based on the gene score and the molecular pathway interconnectivity within the research graph, the processor 104 is further configured to identify a pathway among the one or more molecular pathways associated with the plurality of genes with a highest pathway perturbation score as the perturbed molecular pathway for genes. The perturbed molecular pathway for genes is a molecular pathway which has a highest association with a pathophysiology of the disease of interest or the drug response of the given drug as compared to other molecular pathways associated with the plurality of genes in the research graph. In other words, the determined perturbed molecular pathway by the processor 104 is the highest ranked pathway and has the highest perturbation score. The determined perturbed molecular pathway is utilized to remediate one or more of: the disease of interest and the drug response of the given drug.

In some implementations, the processor 104 is further configured to generate insights to develop new therapeutic strategies and drug targets aimed at modulating an activity of the perturbed molecular pathway and potentially treating the disease of interest or improving the efficacy of the given drug.

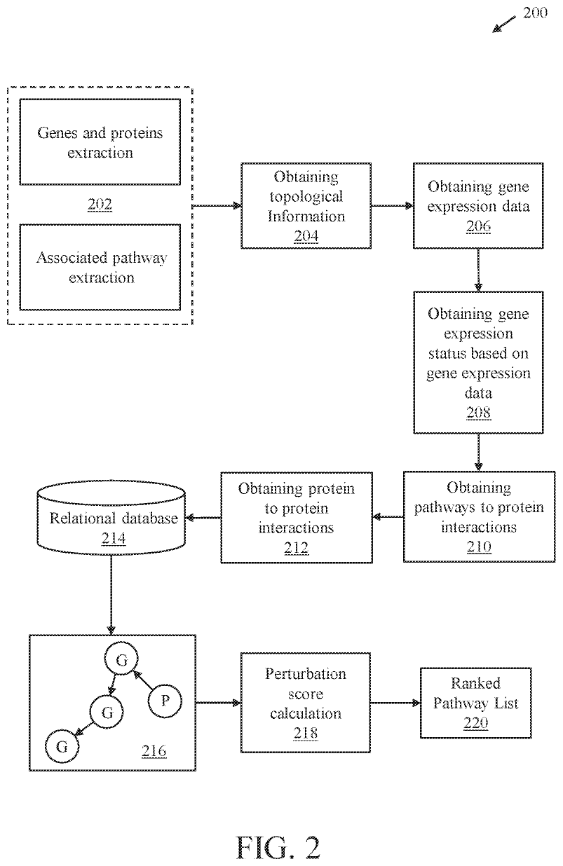

FIG. 2 is a flowchart to rank the molecular pathways based on perturbation score, in accordance with an embodiment of the present disclosure. FIG. 2 is described in conjunction with elements from FIG. 1. With reference to FIG. 2, there is shown a flowchart 200 rank the molecular pathways based on perturbation score. The flowchart 200 includes a series of operations 202 to 218. The operations 202 to 218 are performed by the processor 104.

At operation 202, information related to the plurality of genes and proteins are extracted from the pre-curated database 112. The information may include, but limited to, gene names, gene function, protein names, protein function, molecular interactions, and pathways associated with these genes and proteins. Further, at the operation 202, the one or more pathways associated with the plurality of genes and proteins.

At operation 204, the topological information associated with the plurality of genes and proteins is obtained. The topological information may include network properties such as degree centrality, betweenness centrality, and closeness centrality, which provide information on the importance of a node within a network or graph. In some implementations, the topological information is used to analyze the plurality of genes and proteins to identify the start genes, the end genes, and consequent genes for constructing the research graph.

At operation 206, the gene expression data is obtained from the patient sample. After that, at operation 208, the gene expression status is obtained based on the gene expression data. Based on the gene expression status, the gene score is assigned to each gene of the plurality of genes. The gene score is later used in the calculation of the perturbation score for the disease or the given set of genes.

At operation 210, each of the pathway to protein interactions are obtained. Further, at operation 212, each of the protein to protein interactions are obtained. The interactions obtained in the operations 210 and 212 includes only one directional relationship such as “from” to “to”.

At operation 214, each of the extracted information about the plurality of genes and proteins, the one or more associated pathways, the obtained gene expression status, the gene score, and the obtained interactions between the pathways and proteins are stored in the relation database 120 in a vector form.

At operation 216, using data stored in the relational database 120, the research graph is constructed. Further, the number of initial gene for the one or more molecular pathways (Ig^{N}), predecessors gene count of Ig^{N} (G^{P}), the number of overlapped gene in the research graph (G^{OverlapGene}), the number of the one or more identified sub-networks within the research graph (PC^{N}), the number of molecular pathway inter-connectivity of initial gene network (PC) may be obtained by observing the constructed research graph.

At operation 218, the perturbation score for each molecular pathway is calculated by using Equation 1 or Equation 2. After that, at operation 220, each molecular pathway within the research graph is ranked based on the respective perturbation score.

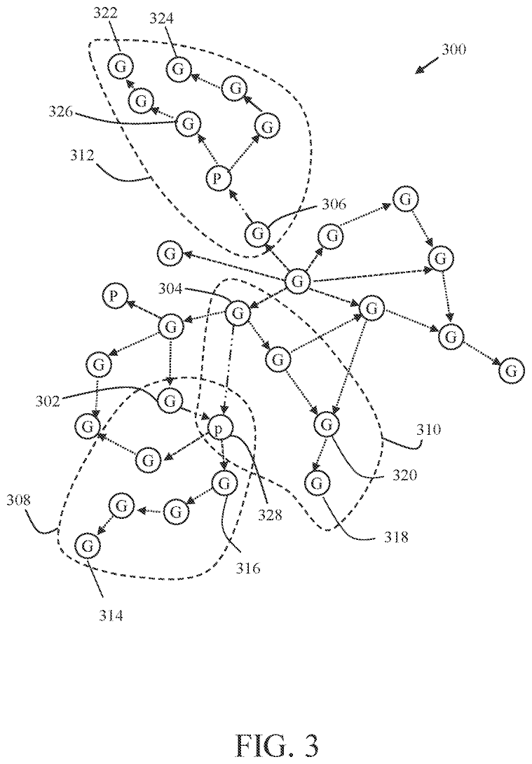

FIG. 3 is an exemplary research graph for a given set of genes, in accordance with an embodiment of the present disclosure. FIG. 3 is described in conjunction with elements from FIGS. 1 and 2. With reference to FIG. 3, there is shown an exemplary research graph 300. The exemplary research graph 300 represents the one or more molecular pathway connectivity of the plurality of genes in the pathophysiological condition. The nodes of the research graph 300 represent genes or proteins, and the edges represent the molecular interactions or relationships between them. The research graph 300 includes three start genes i.e., a first start gene 302, a second start gene 304, and a third start gene 306. Each of the first start gene 302, the second start gene 304, and the third start gene 306 is connected to other genes or protein and the interaction between them is shown by a dash-dot-dot arrow. The research graph 300 further includes three sub-networks i.e., a first sub-network 308, a second sub-network 310, and a third sub-network 312. Each of the first sub-network 308, the second sub-network 310, and the third sub-network 312 includes a start gene, one or more end genes and one or more consequent genes between the start gene and the one or more end genes. For example, the first sub-network 308 starts with the first start gene 302 and ends with a first end gene 314. The first sub-network 308 further includes a first dysregulated gene 316. The second sub-network 310 starts with the second start gene 304 and ends with a second end gene 318. The second sub-network 310 further includes a second dysregulated gene 320. The third sub-network 312 starts with the third start gene 306 and ends with a third end gene 322 and a fourth end gene 324. The third sub-network 312 further includes a third dysregulated gene 326. In addition, the first sub-network 308 and the second sub-network 310 overlap at a molecular pathway 328.

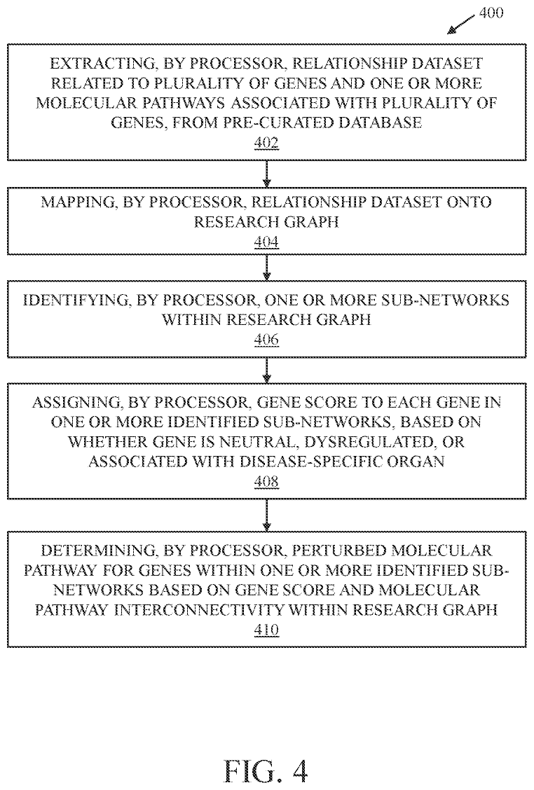

FIG. 4 is a flowchart of a method for identifying molecular pathways perturbed under influence of the given drug or the disease of interest, in accordance with an embodiment of the present disclosure. FIG. 4 is explained in conjunction with elements from FIGS. 1, 2 and 3. With reference FIG. 4, there is shown a flowchart of a method 400. The method 400 is executed at the server 102 (of FIG. 1). The method 400 may include steps 402 to 410.

At step 402, the method 400 includes extracting, by the processor 104, the relationship dataset 114 related to the plurality of genes and the one or more molecular pathways associated with the plurality of genes, from the pre-curated database 112.

At step 404, the method 400 further includes mapping, by the processor 104, the relationship dataset 114 onto the research graph (similar to the research graph 300). The research graph is indicative of the molecular pathway connectivity of the plurality of genes in the pathophysiological condition. In accordance with an embodiment, the mapping of the relationship dataset 114 onto the research graph includes constructing, by the processor 104, the research graph by representing each gene and the one or more associated molecular pathways as the nodes and representing the relationships between the plurality of genes and the one or more associated molecular pathways as the edges in the research graph.

At step 406, the method 400 further includes identifying, by the processor, the one or more sub-networks (similar to the first sub-network 308, the second sub-network 310, and the third sub-network 312 within the research graph 300) within the research graph. Each sub-network includes the start gene (similar to the first start gene 302, the second start gene 304, and the third start gene 306), the one or more end genes (similar to the first end gene 314, the second end gene 318, and the third end gene 322), and the one or more molecular pathways associated with the start gene and the one or more end genes within the research graph. In accordance with an embodiment, the identifying of the one or more sub-networks within the research graph includes identifying, by the processor 104, the one or more start genes within the research graph based on the topological information and the curated pathway data obtained from the curated database 112. The identifying of the one or more sub-networks within the research graph further includes identifying, by the processor 104, the one or more end genes associated with each start gene by following the molecular connectivity in the research graph until the pathway endpoint is reached. The identifying of the one or more sub-networks within the research graph further includes grouping, by the processor 104, each start gene and the associated end genes into the one or more sub-networks based on the molecular connectivity.

At step 408, the method 400 further includes assigning, by the processor 104, the gene score to each gene in the one or more identified sub-networks, based on whether the gene is neutral, dysregulated, or associated with a disease-specific organ. In accordance with an embodiment, the assigning of the gene score to each gene in the one or more identified sub-networks includes obtaining, by the processor 104, the gene expression data from the patient sample, identifying, by the processor 104, the gene expression status based on the gene expression data obtained from the patient sample, and assigning, by the processor 104, the gene score based on the gene expression status. In accordance with an embodiment, the method 400 further includes storing, by the processor 104, the relationship dataset 114 and the gene score of each gene in the relational database 120 in the linear vector form for constructing the research graph.

At step 410, the method 400 further includes determining, by the processor 104, the perturbed molecular pathway for genes within the one or more identified sub-networks based on the gene score and the molecular pathway interconnectivity within the research graph. The perturbed molecular pathway for genes is a molecular pathway which has a highest association with the pathophysiology of the disease of interest or the drug response of the given drug as compared to the other molecular pathways associated with the plurality of genes in the research graph. The determined perturbed molecular pathway is utilized to remediate one or more of: the disease of interest and the drug response of the given drug. in accordance with an embodiment, the determining of the perturbed molecular pathway for genes within the one or more identified sub-networks based on the gene score and the molecular pathway interconnectivity within the research graph includes identifying, by the processor 104, the pathway among the one or more molecular pathways associated with the plurality of genes with the highest pathway perturbation score as the perturbed molecular pathway for genes.

In accordance with an embodiment, the method 400 further includes calculating, by the processor 104, the pathway perturbation score for each molecular pathway within the one or more identified sub-networks based on the gene score and the molecular pathway interconnectivity within the one or more identified sub-networks.

In accordance with an embodiment, the method 400 further includes ranking, by the processor 104, the one or more associated molecular pathways within each identified sub-network based on the respective pathway perturbation score. The higher ranked pathway is more perturbed than the lower ranked pathway.

In accordance with an embodiment, the method 400 further includes generating, by the processor 104, insights to develop new therapeutic strategies and drug targets aimed at modulating an activity of the perturbed molecular pathway and potentially treating the disease of interest or improving the efficacy of the given drug.

The system 100 and the method 400 of the present disclosure utilizes the pre-curated database 112 and the research graph to efficiently identify the molecular pathways perturbed by the given drug or disease. This reduces the time and resources required for identifying perturbed molecular pathways compared to traditional experimental approaches. By assigning the gene scores and considering molecular pathway interconnectivity within the research graph, the system 100 and method 400 accurately determines the perturbed molecular pathway. This ensures that the remediation strategies are focused on the most relevant molecular pathways, increasing the likelihood of success.

Further, the system 100 and the method 400 identifies the molecular pathways perturbed under the influence of a specific disease or drug, allowing doctors to develop personalized treatment plans that target those pathways. By identifying the molecular pathways perturbed under the influence of the specific disease or drug, the system 100 and the method 400 provide insights into the underlying mechanisms of drug action and disease pathology, which may help researchers develop more effective treatments. For example, if a particular drug is found to perturb a specific molecular pathway, researchers develop new drugs that target that pathway more specifically. This may lead to better patient outcomes and fewer adverse drug reactions, which ultimately reduce healthcare costs.

Furthermore, the system 100 and the method 400 may also be applied to a wide range of diseases and drugs, as it is not limited to any particular disease or drug. Thus, making it a versatile tool for drug discovery and personalized medicine. The versatility of the system 100 and method 400 lies in its ability to analyse complex molecular interactions and identify key targets for drug development and personalized medicine, regardless of the specific disease or drug being studied. For example, if a new drug is developed and its mechanism of action is not fully understood, the method 400 is used to identify the molecular pathways that are affected by the drug and predict its potential side effects. Similarly, if a disease is poorly understood or has complex aetiology, the method 400 is used to identify the molecular pathways that are involved in the disease and suggest potential therapeutic targets.

### Example 1

In Example 1, a total of 16 genes were inputted as an input gene list in the processor 104. The input gene list includes ‘KDR’, ‘RET’, ‘FGFR1’, ‘KDR’, ‘APP’, ‘NTRK1’, ‘EGFR’, ‘SQSTM1’, ‘NUFIP2’, ‘GNB1’, ‘RPL22’, ‘SYNC’, ‘RIP’, ‘RPN1’, ‘RPL36’, and ‘C1QTNF9B’. The processor 104 extracted relationship data related to the one or more pathways associated with each gene in the input gene list from the pre-curated database 112. After that, the processor 104 map each gene of the input gene list as nodes onto a research graph and the relation data as edges onto the research graph. Further, the processor 104 identified one or more sub-networks within the research graph, and then assigned a gene score to each gene in the identified sub-networks, based on whether a gene is neutral, dysregulated, or associated with a disease-specific organ. Finally, the processor 104 calculated the perturbation score for each gene in the input gene list and then rank the perturbed molecular pathway for the input gene list from a highest association to a lowest association with a pathophysiology of the disease or the drug response.

TABLE 1
perturbed pathways of the input gene list in descending order

| | | | | | IgC = Start |
|---|---|---|---|---|---|
| | Overlap | | Ig = Start | IgN = | gene overlap |
| | Gene | Overlap | Gene of the | Start Gene | with overlap |
| Pathway name | count | Gene name | pathways | count | gene |
| MAPK | 2 | ‘EGFR’, | MAPK | 1 | 0 |
| signalling | | ‘FGFR1’ | | | |
| pathway | | | | | |
| MAPK1/MAPK3 | 3 | ‘EGFR’, | MAPK1, | 2 | 0 |
| signalling | | ‘RET’, | MAPK3 | | |
| | | ‘FGFR1’ | | | |
| Angiogenesis | 2 | ‘KDR’, | ‘FGFR1’ | 1 | 1 |
| pathway | | ‘FGFR1’ | | | |
| Negative | 2 | ‘EGFR’, | PI3K, AKT | 2 | 0 |
| regulation of the | | ‘FGFR1’ | | | |
| PI3K/AKT | | | | | |
| network | | | | | |
| Signalling by | 2 | ‘RPL36’, | ROBO | 2 | 0 |
| ROBO receptors | | ‘RPL22’ | | | |
| PI3K/AKT | 3 | ‘EGFR’, | PI3K, AKT | 2 | 0 |
| Signalling in | | ‘FGFR1’ | | | |
| Cancer | | | | | |
| Proteoglycans in | 2 | ‘KDR’, | CD44, | 2 | 0 |
| cancer | | ‘EGFR’, | IGF1, | | |
| | | ‘FGFR1’ | HER2 | | |
| Signalling | 2 | ‘EGFR’, | PI3KR1, | 6 | 0 |
| Pathways in | | ‘FGFR1’ | PI3KR2, | | |
| Glioblastoma | | | PI3KCA | | |
| Constitutive | 2 | ‘EGFR’, | PI3K | 1 | 0 |
| Signalling by | | ‘FGFR1’ | | | |
| Aberrant | | | | | |

| | GP = | | |
|---|---|---|---|
| | Successor | GS = | Pathway |
| | gene count | Gene | perturbation |
| Pathway name | of IgN | Score | Score |
| MAPK | 323 | 2 | 6480.053 |
| signalling | | | |
| pathway | | | |
| MAPK1/MAPK3 | 278 | 2 | 5600.079 |
| signalling | | | |
| Angiogenesis | 174 | 2 | 3675.053 |
| pathway | | | |
| Negative | 111 | 2 | 2260.053 |
| regulation of the | | | |
| PI3K/AKT | | | |
| network | | | |
| Signalling by | 221 | 1 | 2230.053 |
| ROBO receptors | | | |
| PI3K/AKT | 102 | 2 | 2080.053 |
| Signalling in | | | |
| Cancer | | | |
| Proteoglycans in | 198 | 1 | 2010.079 |
| cancer | | | |
| Signalling | 81 | 2 | 1740.053 |
| Pathways in | | | |
| Glioblastoma | | | |
| Constitutive | 75 | 2 | 1520.053 |
| Signalling by | | | |
| Aberrant | | | |

Table 1 includes the name of biological pathways from various publicly available databases in first column. Table 1 further includes overlap gene count, overlap gene name, start gene of the pathways, start gene count, start gene overlap with overlap gene, successor gene count of start gene count, gene scores and perturbation score based on Equation 2.

Modifications to embodiments of the present disclosure described in the foregoing are possible without departing from the scope of the present disclosure as defined by the accompanying claims. Expressions such as “including”, “comprising”, “incorporating”, “have”, “is” used to describe and claim the present disclosure are intended to be construed in a non-exclusive manner, namely allowing for items, components or elements not explicitly described also to be present. Reference to the singular is also to be construed to relate to the plural. The word “exemplary” is used herein to mean “serving as an example, instance or illustration”. Any embodiment described as “exemplary” is not necessarily to be construed as preferred or advantageous over other embodiments and/or to exclude the incorporation of features from other embodiments. The word “optionally” is used herein to mean “is provided in some embodiments and not provided in other embodiments”. It is appreciated that certain features of the present disclosure, which are, for clarity, described in the context of separate embodiments, may also be provided in combination in a single embodiment. Conversely, various features of the present disclosure, which are, for brevity, described in the context of a single embodiment, may also be provided separately or in any suitable combination or as suitable in any other described embodiment of the disclosure.

1. A method for identifying molecular pathways perturbed under influence of a given drug or a disease of interest, the method comprising:
extracting, by a processor, a relationship dataset related to a plurality of genes and one or more molecular pathways associated with the plurality of genes, from a pre-curated database;
mapping, by the processor, the relationship dataset onto a research graph, wherein the research graph is indicative of a molecular pathway connectivity of the plurality of genes in a pathophysiological condition;
identifying, by the processor, one or more sub-networks within the research graph, wherein each sub-network comprises a start gene, one or more end genes, and one or more molecular pathways associated with the start gene and the one or more end genes within the research graph;
assigning, by the processor, a gene score to each gene in the one or more identified sub-networks, based on whether a gene is neutral, dysregulated, or associated with a disease-specific organ; and
determining, by the processor, a perturbed molecular pathway for genes within the one or more identified sub-networks based on the gene score and a molecular pathway interconnectivity within the research graph,
wherein the perturbed molecular pathway for genes is a molecular pathway which has a highest association with a pathophysiology of the disease of interest or the drug response of the given drug as compared to other molecular pathways associated with the plurality of genes in the research graph, and
wherein the determined perturbed molecular pathway is utilized to remediate one or more of: the disease of interest and the drug response of the given drug.

2. The method of claim 1, wherein the mapping of the relationship dataset onto the research graph comprises: constructing, by the processor, the research graph by representing each gene and the one or more associated molecular pathways as nodes and representing the relationships between the plurality of genes and the one or more associated molecular pathways as edges in the research graph.

3. The method of claim 1, wherein the assigning of the gene score to each gene in the one or more identified sub-networks comprises:
obtaining, by the processor, gene expression data from a patient sample;
identifying, by the processor, gene expression status based on the gene expression data obtained from the patient sample; and
assigning, by the processor, the gene score based on the gene expression status.

4. The method of claim 1, further comprising storing, by the processor, the relationship dataset and the gene score of each gene in a relational database in a linear vector form for constructing the research graph.

5. The method of claim 1, wherein the identifying of the one or more sub-networks within the research graph comprises:
identifying, by the processor, one or more start genes within the research graph based on topological information and curated pathway data obtained from the curated database;
identifying, by the processor, the one or more end genes associated with each start gene by following the molecular connectivity in the research graph until a pathway endpoint is reached; and
grouping, by the processor, each start gene and the associated end genes into the one or more sub-networks based on the molecular connectivity.

6. The method of claim 1, wherein the one or more identified sub-networks comprises a homogeneous network, a heterogeneous network, a heterogeneous multi-layered network, or a combination thereof.

7. The method of claim 1, further comprising calculating, by the processor, a pathway perturbation score for each molecular pathway within the one or more identified sub-networks based on the gene score and the molecular pathway interconnectivity within the one or more identified sub-networks.

8. The method of claim 7, further comprising ranking, by the processor, the one or more associated molecular pathways within each identified sub-network based on the respective pathway perturbation score, wherein a higher ranked pathway is more perturbed than a lower ranked pathway.

9. The method of claim 1, wherein the determining of the perturbed molecular pathway for genes within the one or more identified sub-networks based on the gene score and the molecular pathway interconnectivity within the research graph comprises: identifying, by the processor, a pathway among the one or more molecular pathways associated with the plurality of genes with a highest pathway perturbation score as the perturbed molecular pathway for genes.

10. The method of claim 1, further comprising generating, by the processor, insights to develop new therapeutic strategies and drug targets aimed at modulating an activity of the perturbed molecular pathway and potentially treating the disease of interest or improving the efficacy of the given drug.

11. A system for identifying molecular pathways perturbed under influence of a given drug or a disease of interest, the system comprising:
a processor configured to:
extract a relationship dataset related to a plurality of genes and one or more molecular pathways associated with the plurality of genes, from a pre-curated database;
map the relationship dataset onto a research graph, wherein the research graph is indicative of a molecular pathway connectivity of the plurality of genes in a pathophysiological condition;
identify one or more sub-networks within the research graph, wherein each sub-network comprises a start gene, one or more end genes, and one or more molecular pathways associated with the start gene and the one or more end genes within the research graph;
assign a gene score to each gene in the one or more identified sub-networks, based on whether a gene is neutral, dysregulated, or associated with a disease-specific organ; and
determine a perturbed molecular pathway for genes within the one or more identified sub-networks based on the gene score and a molecular pathway interconnectivity within the research graph,
wherein the perturbed molecular pathway for genes is a molecular pathway which has a highest association with a pathophysiology of the disease of interest or the drug response of the given drug as compared to other molecular pathways associated with the plurality of genes in the research graph, and
wherein the determined perturbed molecular pathway is utilized to remediate one or more of: the disease of interest and the drug response of the given drug.

12. The system of claim 11, wherein, in order to map the relationship dataset onto the research graph, the processor is further configured to construct the research graph by representing each gene and the one or more associated molecular pathways as nodes and representing the relationships between the plurality of genes and the one or more associated molecular pathways as edges in the research graph.

13. The system of claim 11, wherein, in order to assign the gene score to each gene in the one or more identified sub-networks, the processor is further configured to:
obtain gene expression data from a patient sample;
identify gene expression status based on the gene expression data obtained from the patient sample; and
assign the gene score based on the gene expression status.

14. The system of claim 11, wherein the processor is further configured to store the relationship dataset and the gene score of each gene in a relational database in a linear vector form for constructing the research graph.

15. The system of claim 11, wherein, in order to identify the one or more sub-networks within the research graph, the processor is further configured to:
identify one or more start genes within the research graph based on topological information and curated pathway data obtained from the curated database;
identify the one or more end genes associated with each start gene by following the molecular connectivity in the research graph until a pathway endpoint is reached; and
group each start gene and the one or more associated end genes into the one or more sub-networks based on the molecular connectivity.

16. The system of claim 11, wherein the one or more identified sub-networks comprises a homogeneous network, a heterogeneous network, a heterogeneous multi-layered network, or a combination thereof.

17. The system of claim 11, wherein the processor is further configured to calculate a pathway perturbation score for each molecular pathway within the one or more identified sub-networks based on the gene score and the molecular pathway interconnectivity within the one or more identified sub-networks.

18. The system of claim 17, wherein the processor is further configured to rank the one or more associated molecular pathways within each identified sub-network based on the respective pathway perturbation score, and wherein a higher ranked pathway is more perturbed than a lower ranked pathway.

19. The system of claim 11, wherein, in order to determine the perturbed molecular pathway for genes within the one or more identified sub-networks based on the gene score and the molecular pathway interconnectivity within the research graph, the processor is further configured to identify a pathway among the one or more molecular pathways associated with the plurality of genes with a highest pathway perturbation score as the perturbed molecular pathway for genes.

20. The system of claim 11, wherein the processor is further configured to generate insights to develop new therapeutic strategies and drug targets aimed at modulating an activity of the perturbed molecular pathway and potentially treating the disease of interest or improving the efficacy of the given drug.
